(19) United States
(12) Patent Application Publication
(10) Pub. No.: US 2018/0177527 A1
(43) Pub. Date: Jun. 28, 2018

(54) APPARATUS FOR EXTRACTING PARASITES

(71) Applicants: Tomer COHEN, Haifa (IL); Emanuel COHEN, Kiryat Gat (IL)

(72) Inventors: Tomer COHEN, Haifa (IL); Emanuel COHEN, Kiryat Gat (IL)

(21) Appl. No.: 15/387,982

(22) Filed: Dec. 22, 2016

(51) Int. Cl. A61B 17/50

(52) U.S. Cl. CPC A61B 17/50; A61B 2017/505

(57) ABSTRACT

An apparatus for extracting parasites from a rectum includes a shaft and a guard portion. The shaft with a distal portion is configured to be inserted through an anus into the rectum, the distal portion of the shaft including a plurality of protrusions or a plurality of grooves designed to catch and hold parasites when removed from the rectum, the shaft further including a proximal portion designed to serve as a handle. The guard portion is positioned on the shaft between the proximal portion and the distal portion of the shaft to prevent the distal portion from being inserted into the rectum further than a predefined distance.

### FIELD OF THE INVENTION

The present invention relates to parasitic infections, and particularly relates to an apparatus for extracting parasites (e.g., pinworms) from a rectum.

### BACKGROUND OF THE INVENTION

Parasitic infections in a subject, such as a human subject, may include a pinworm infection. Pinworm infection, or enterobiasis, is quite common in humans, particularly in children. The infection may be spread by the ingestion of pinworm eggs. The pinworm eggs may hatch in the intestinal tract into pinworms, or enterobius vermicularis, and grow within the intestine.

Pinworms are typically very soft and visible to the naked eye. However, pinworm eggs are on the order of 50 μm in size and are translucent. The surface of pinworm eggs is sticky when laid. They may be deposited in and around the anus, perineum and rectal lumen near the anus. In females, the pinworms and pinworm eggs may be depositing in and around the vagina.

The pinworms may replicate in the lower intestinal tract by laying eggs around the rectum or anus in the infected subject. The activity and movement of the pinworms around the anus and surrounding region such as the perineum may result in pruritus ani and/or perineal pruritus, or severe itching, pain, or feelings of a tickling or crawling sensation in and around the affected regions. These sensations may cause the subject to scratch the affected regions. As a result, pinworm eggs may stick to the fingers of the subject and/or collect under the fingernails of the subject, for example. These pinworm eggs may be transferred and ingested by other humans, thus spreading the pinworm infection.

Pinworms may be more active at night when the subject may be asleep. The resultant abdominal pain and/or itch sensations from the pinworm infection may wake up the subject. The body of pinworms may be soft in texture. Thus, pinworms may be killed by cutting, smearing, and/or smashing the bodies of the pinworms. Removing visible pinworms and/or smearing the pinworms in the anus and/or in the rectal lumen with a tool or brush inserted into the anus may immediately reduce the discomfort due to pruritus ani and/or perineal pruritus.

Medications may be used to alleviate pinworm infection. For example, the most effective medications may include albendazole and mebendazole. However, administering mebendazole, for example, to a patient may result in side effects, such as diarrhea and abdominal pain. Thus, if the subject, such as a child, for example, exhibits symptoms of pinworm infection but no pinworms have been observed, it may be desirable to have an apparatus for extracting and detecting pinworms and/or pinworm eggs from the intestinal tract to verify if a pinworm infection may exist prior to administering medications to avoid any unnecessary side effects.

Thus, it may be advantageous to have an apparatus for extracting parasites so as to detect pinworm infection in a subject prior to administering anti-pinworm medications.

### SUMMARY OF THE INVENTION

There is thus provided, in accordance with some embodiments of the present invention, an apparatus for extracting parasites from a rectum that may include a shaft and a guard portion. The shaft may include a distal portion configured to be inserted through an anus into the rectum, the distal portion of which shaft may include a plurality of protrusions or a plurality of grooves designed to catch and hold parasites when removed from the rectum, and the shaft may further include a proximal portion designed to serve as a handle. The guard portion may be positioned on the shaft between the proximal portion and the distal portion of the shaft to prevent the distal portion from being inserted into the rectum further than a predefined distance.

Furthermore, in accordance with some embodiments of the present invention, the plurality of protrusions or the plurality of grooves may be designed to catch and hold parasites selected from the group consisting of pinworms and pinworm eggs.

Furthermore, in accordance with some embodiments of the present invention, the plurality of protrusions may include a plurality of spherical caps.

Furthermore, in accordance with some embodiments of the present invention, the plurality of protrusions may include a plurality of bristles.

Furthermore, in accordance with some embodiments of the present invention, the plurality of bristles may include a respective plurality of parasite collector tabs, where extraction of the shaft from the rectum may cause the plurality of parasite collector tabs to contact walls of the rectum and collect parasites.

Furthermore, in accordance with some embodiments of the present invention, the plurality of protrusions may be wound in a helical coil configuration around the shaft.

Furthermore, in accordance with some embodiments of the present invention, the shaft, the plurality of protrusions, and the plurality of grooves may be formed from materials consisting of silicone, rubber, plastic, and nylon.

Furthermore, in accordance with some embodiments of the present invention, the shaft may be molded to form the plurality of protrusions or the plurality of grooves.

Furthermore, in accordance with some embodiments of the present invention, the apparatus may be configured to be stored in a sterile cap.

Furthermore, in accordance with some embodiments of the present invention, the plurality of protrusions or the plurality of grooves may include a lubricant to ease insertion of the shaft into the rectum.

Furthermore, in accordance with some embodiments of the present invention, the guard portion may include a disk stopper.

Furthermore, in accordance with some embodiments of the present invention, the guard portion may be formed from a change in a diameter of the shaft.

Furthermore, in accordance with some embodiments of the present invention, the apparatus may include a vial, and the proximal portion of the shaft may include a cap configured to be attached to an open end of the vial.

Furthermore, in accordance with some embodiments of the present invention, the vial may include a reagent which changes color to indicate a presence of extracted parasites in the plurality of protrusions or the plurality of grooves.

Furthermore, in accordance with some embodiments of the present invention, the vial may include at least one lens configured to magnify the extracted parasites in the plurality of protrusions or the plurality of grooves.

Furthermore, in accordance with some embodiments of the present invention, the vial may include a coloring dye for dying the extracted parasites in the plurality of protrusions or the plurality of grooves to enhance observation of the extracted parasites using the at least one lens.

Furthermore, in accordance with some embodiments of the present invention, the shaft may include the plurality of protrusions or the plurality of grooves configured to kill parasites in the rectum by repeatedly inserting and withdrawing the shaft from the rectum.

Furthermore, in accordance with some embodiments of the present invention, the shaft may include a texture where the plurality of protrusions or the plurality of grooves are configured to extract the parasites.

Furthermore, in accordance with some embodiments of the present invention, the apparatus may include a bulb attached to the proximal portion of the shaft, and the bulb may be configured to create a vacuum at an aperture of a bore formed in the shaft which draws parasites though the aperture into the bore.

Furthermore, in accordance with some embodiments of the present invention, the apparatus may include a bulb attached to the proximal portion of the shaft, and the bulb may be configured to change pressure within a bore in the shaft, the changing pressure causing a change in a diameter of the shaft.

### BRIEF DESCRIPTION OF THE DRAWINGS

In order for the present invention, to be better understood and for its practical applications to be appreciated, the following figures are provided and referenced hereafter. It should be noted that the figures are given as examples only and in no way limit the scope of the invention. Like components are denoted by like reference numerals.

FIG. 1A schematically illustrates a first embodiment of an apparatus for extracting parasites, in accordance with some embodiments of the present invention;

FIG. 1B schematically illustrates a second embodiment of an apparatus for extracting parasites, in accordance with some embodiments of the present invention;

FIG. 1C schematically illustrates a third embodiment of an apparatus for extracting parasites, in accordance with some embodiments of the present invention;

FIG. 1D schematically illustrates a fourth embodiment of an apparatus for extracting parasites, in accordance with some embodiments of the present invention;

FIG. 1E schematically illustrates a fifth embodiment of an apparatus for extracting parasites, in accordance with some embodiments of the present invention;

FIG. 1F schematically illustrates a sixth embodiment of an apparatus for extracting parasites, in accordance with some embodiments of the present invention;

FIG. 2A schematically illustrates the fifth embodiment of apparatus for extracting parasites prior to insertion into a rectum, in accordance with some embodiments of the present invention;

FIG. 2B schematically illustrates the fifth embodiment of apparatus for extracting parasites after withdrawal from rectum with pinworm eggs caught in parasite collector tabs, in accordance with some embodiments of the present invention;

FIG. 3A schematically illustrates an apparatus for extracting parasites and a vial for storing the apparatus after use, in accordance with some embodiments of the present invention;

FIG. 3B schematically illustrates an apparatus for extracting parasites inserted into a vial after use, in accordance with some embodiments of the present invention;

FIG. 4A schematically illustrates a seventh embodiment of an apparatus for extracting parasites including a bulb, in accordance with some embodiments of the present invention; and

FIG. 4B schematically illustrates an eighth embodiment of an apparatus for extracting parasites including bulb, in accordance with some embodiments of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, use of the conjunction “or” as used herein is to be understood as inclusive (any or all of the stated options).

According to some embodiments of the present invention, an apparatus for extracting parasites from a rectum is described hereinafter. The apparatus for extracting parasites may include a shaft, or a rod, typically cylindrical having a distal portion configured to be inserted through an anus into the rectum. The distal portion of the shaft may include a plurality of protrusions or a plurality of grooves designed to catch and hold parasites when removed from the rectum. The shaft may further include a proximal portion designed to serve as a handle. A guard portion may be positioned on the shaft between the proximal portion and the distal portion to prevent the distal portion from being inserted into the rectum, or a lumen of the rectum, further than a predefined distance.

In some embodiments of the present invention, the apparatus for extracting parasites may be used for extracting parasites such as pinworms, or enterobius vermicularis. In other embodiments, the apparatus for extracting parasites may be used for extracting parasites, such as pinworms eggs. Other embodiments of the present invention may be directed to extracting any other species or types of parasite from the anus and/or the rectum, for example, female genital worms or parasites.

The apparatus for extracting parasites may be initially stored in a sterile environment prior to use. After the apparatus for extracting parasites is used to extract and collect parasites, such as pinworms and/or pinworm eggs from the rectum of the subject via the anus, the used apparatus for extracting parasites may be stored in a cap, vial, container, or vessel for storing the collected sample. In various embodiments, the vial may include at least one lens to magnify the collected sample. In various embodiments, the vial may include a fluid for staining the pinworm and/or pinworm eggs. In various embodiments, the vial may include a detecting solution, or reagent, which may change color when pinworms and/or pinworm eggs are present in the collected sample.

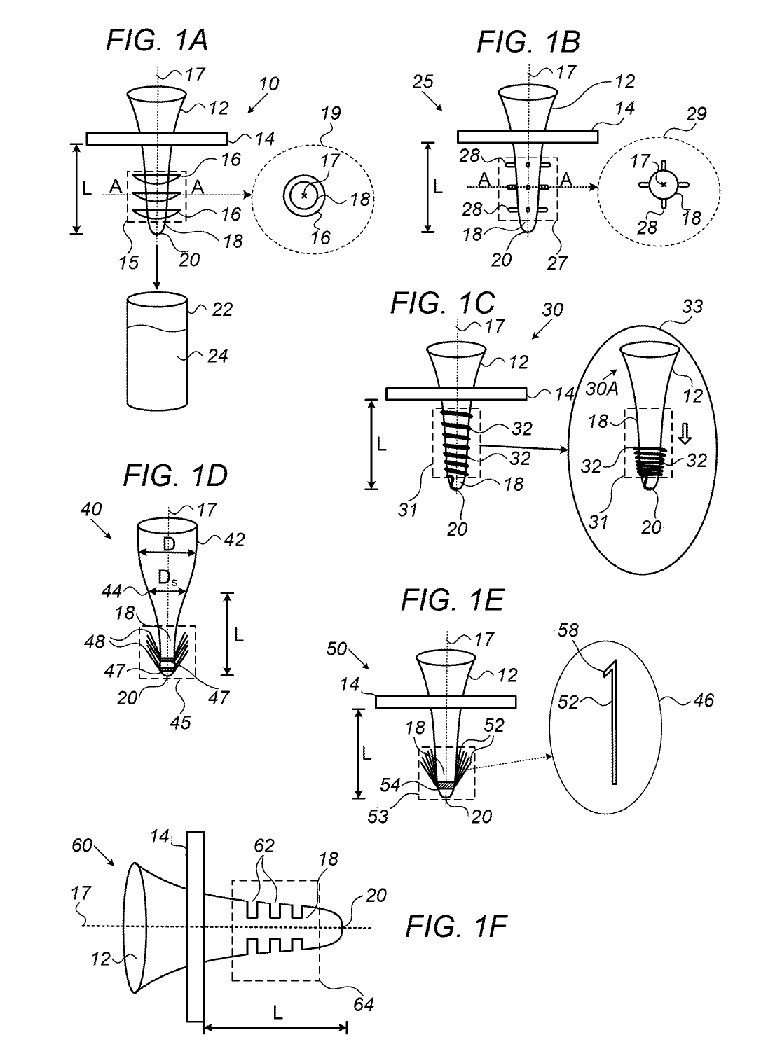

FIGS. 1A-1F schematically illustrate various embodiments of an apparatus for extracting parasites, in accordance with some embodiments of the present invention.

FIG. 1A schematically illustrates a first embodiment of an apparatus 10 for extracting parasites, in accordance with some embodiments of the present invention. Apparatus 10 for extracting parasites may include a shaft 18, or a rod 18, substantially cylindrical with a distal portion 20 configured for insertion through the anus into the lumen of a rectum of the subject, such as for example, possessing a rounded, polished and/or smooth distal portion 20. A proximal portion of shaft 18 may include handle 12. Handle 12 may be used to prevent the user's hands and/or fingers from contacting the pinworm and/or pinworm eggs when collecting the sample, and thus preventing infection of the user. A guard portion, such as a disk stopper 14, may be positioned on shaft 18 between handle 12 and distal portion 20 to prevent the distal portion from being inserted further than a predefined distance L into the rectal lumen. During insertion of shaft 18 into the rectum of the subject via the anus, disk stopper 14 may be stopped by the anal walls so as to prevent shaft 18 from being inserted further into the anus. L may be, for example, about 2.5-3 cm.

Shaft 18 in a region 15 at distal portion 20 may include a plurality of flexible protrusions 16 such as a plurality of flexible spherical caps 16, for parasite extraction which may be symmetrically positioned around a longitudinal axis 17 of shaft 18. The concavity of flexible spherical caps 16 are shown upward such that flexible spherical caps 16, which may facilitate pinworm and/or pinworm egg collection when shaft 18 is extracted from the rectum. The concavity of flexible spherical caps 16 may include any suitable curvature and/or shape.

Flexible spherical cap 16 may be formed from silicone, rubber, plastic, nylon, etc. In some embodiments, the plurality of flexible spherical caps 16 may be formed by molding shaft 18 concurrently with the plurality of flexible spherical cap 16. In other embodiments, the plurality of flexible spherical caps 16 may be affixed to shaft 18 by gluing, for example. In some embodiments, flexible spherical cap 16 may be shaped as a flat, flexible disc without curvature.

The plurality of flexible spherical caps 16 may be configured to contact walls of the rectal lumen and to catch and hold parasites. The plurality of flexible spherical caps 16 may have a diameter substantially less than the maximum diameter of the distended rectum or about 1.5-2 cm in adults. The rectum is normally collapsed and becomes distended when feces traverse the rectum, for example.

Pinworm eggs may adhere to the one or more flexible spherical caps 16 after insertion into the rectum. The plurality of flexible spherical caps 16 may also be used to smash, smear, cut or otherwise destroy live pinworms during insertion into the rectum so as to kill the pinworms. The plurality of flexible spherical caps 16 may be sufficiently soft and flexible so as to bend or otherwise comply during insertion through the anus into the lumen of the rectum, so as not to cause any injury. A cross-section 19 of apparatus 10 for extracting parasites across the line marked A-A, is shown, perpendicular to cylindrical axis 17, with shaft 18 and concentric flexible spherical caps 16 shown as an annular ring with shaft 18 (e.g., a cross-section cut of a flexible spherical cap 16 across the line marked A-A) passing through the annular ring center.

In some embodiments of the present invention, the plurality of flexible spherical caps 16 may not be rigidly fixed to shaft 18, and may be configured to collapse one into the other upon extraction of shaft 18 from the rectum so as to capture the pinworms and/or pinworm eggs between each of the flexible spherical caps 16.

In some embodiments of the present invention, apparatus 10 for extracting parasites may be stored and/or transported in a sterile protection cap 22, which is configured to maintain sterility of the shaft 18, or at least the distal portion of the shaft, with flexible spherical caps 16. Flexible spherical caps 16 may be lubricated by a lubricant. Alternatively and/or additionally, cap 22 may include a lubricant such as a lubricant gel and/or fluid 24 to ease insertion of distal portion 20 of shaft 18 into the rectum of the subject and/or maintain sterility. Lubricant gel and/or fluid 24 may include, for example, K-Y® jelly, Vaseline®, etc. Each of the alternative embodiments of apparatus for extracting parasites shown herein such as in FIGS. 1B-1F, for example, may use sterile protection cap 22 and lubricants as described here.

In some embodiments, the collected sample may be stored in cap 22 for transport to a laboratory, for example, for identification of parasites collected on the plurality of flexible protrusions 16. Note that cap 22 may be adopted to store the collected sample for any of the following embodiments of apparatuses for extracting parasites, and is not limited to the embodiment shown in FIG. 1A.

FIG. 1B schematically illustrates a second embodiment of an apparatus 25 for extracting parasites, in accordance with some embodiments of the present invention. Apparatus 25 for extracting parasites may include shaft 18 with distal portion 20 configured for insertion through the anus into the lumen of a rectum of the subject. A proximal portion of shaft 18 may include handle 12. Guard portion 14, such as disk stopper 14, may be positioned on shaft 18 between handle 12 and distal portion 20 to prevent the distal portion from being inserted further than a predefined distance L into the rectal lumen. Distance L is typically on the order of 2.5-3 cm for example.

Shaft 18 in a region 27 at or near distal portion 20 may include a plurality of flexible protrusions 28 symmetrically positioned around cylindrical axis 17 passing through shaft 18 proximate to distal portion 20. The plurality of flexible protrusions 28 may be formed from silicone, rubber, plastic, and/or nylon, for example. In some embodiments, the plurality of flexible protrusions 28 may be formed by molding shaft 18 concurrently with the plurality of flexible protrusions 28.

A cross-section 29 of apparatus 25 for extracting parasites across the line marked A-A is shown perpendicular to cylindrical axis 17 with shaft 18 and four flexible protrusions 28 positioned symmetrically around shaft 18, which is shown merely for conceptual clarity and not by way of limitation of the embodiments of the present invention. Any suitable number of flexible protrusions 28 may be used to collect and/or extract pinworms and/or pinworm eggs. Apparatus 25 for extracting parasites may be stored and/or transported in a sterile protection cap 22 with lubricant gel and/or fluid 24 as previously described. The sum of the length of the two flexible protrusions 28 and the diameter of shaft 18 may be substantially less than the maximum diameter of the distended rectum or about 1.5-2 cm in adults. Flexible protrusions 28 may be of any suitable length and shape. Flexible protrusions 28 may be grouped, for example, in groups of long and short lengths or any permutation thereof. In some embodiments, flexible protrusions 28 may be shaped to have the tips of flexible protrusions 28 bent upward, downward, or in any suitable shape or manner, for example. In other embodiments, flexible protrusions 28 may be shaped to optimize the collection of parasites when inserted into the rectum.

In each of the alternative embodiments shown in FIGS. 1B-1E, pinworm eggs may stick to the plurality of flexible protrusions, or alternate embodiments thereof, after insertion into the rectum via the anus. The plurality of flexible protrusions may also be used to smash, smear and/or cut live pinworms during insertion into the rectum so as to kill the pinworms. The plurality of flexible protrusions may be sufficiently flexible so as to bend during insertion through the anus into the lumen of the rectum so as not to cause injury. The flexible protrusions may be configured to contact walls of the rectal lumen and to extract parasites.

FIG. 1C schematically illustrates a third embodiment of an apparatus 30 for extracting parasites, in accordance with some embodiments of the present invention. Apparatus 30 for extracting parasites may include shaft 18 with distal portion 20 configured for insertion through the anus into the lumen of a rectum of the subject. A proximal portion of shaft 18 may include handle 12. Guard portion 14, such as disk stopper 14, may be positioned on shaft 18 between handle 12 and distal portion 20 to prevent the distal portion from being inserted further than a predefined distance L into the rectal lumen. Distance L is typically on the order of 2.5-3 cm.

Shaft 18 in a region 31 at or near distal portion 20 may include a plurality of flexible protrusions 32 wound in a helical coil configuration around shaft 18 symmetrically about cylindrical axis 17 and proximate to distal portion 20. The plurality of flexible protrusions 32 may be formed from silicone, rubber, plastic, and/or nylon, for example. The plurality of flexible protrusions 32 may be soft and flexible when inserted and/or withdrawn from the rectum, yet may be rigid enough to maintain contact with the rectal walls. In some embodiments, the plurality of flexible protrusions 32 may be formed by molding shaft 18 concurrently with the plurality of flexible protrusions 32 in the helical coil configuration.

FIG. 1C also includes an inset 33 which illustrates an apparatus 30A for extracting parasites, which is the same structure as apparatus 30. However, in some embodiments, the plurality of flexible protrusions 32 wound in a helical coil configuration around shaft 18 are not fixed to shaft 18 except at point near distal portion 20 of shaft 18. As a result, after insertion of shaft 18 in the rectum and parasites collected on the plurality of flexible protrusions 32 wound in a helical coil, upon withdrawal of shaft 18, adjacent flexible protrusions 32 may collapse one on the other so as to trap the collected parasites between the adjacent flexible protrusions 32 as shown in region 31.

FIG. 1D schematically illustrates a fourth embodiment of an apparatus 40 for extracting parasites, in accordance with some embodiments of the present invention. Apparatus 40 for extracting parasites may include a tapering shaft 18 with distal portion 20 configured for insertion through the anus into the lumen of a rectum of the subject. The diameter of the shaft at the distal portion may be, for example, about 3-4 mm in diameter. A proximal portion of shaft 18 may include handle 12. A guard portion 44 may be integrally formed by grading or changing the shaft diameter D along the length of shaft 18. Guard portion 44 may be effectively formed where the shaft diameter D becomes larger than a stopper diameter Ds typically 1-2 cm, for example. For D larger than Ds, shaft 18 may become too wide to be inserted into the rectum and may be stopped by the anal walls. Guard portion 44 may behave similarly to disk stopper 14 where the position of stopper diameter Ds along cylindrical axis 17 of apparatus 40 for extracting parasites prevents the distal portion from being inserted further than a predefined distance L into the rectal lumen. Distance L is typically on the order of 2.5-3 cm.

Shaft 18 in a region 45 at or near distal portion 20 may include a plurality of flexible protrusions such as a plurality of flexible bristles 48 attached to one or a plurality rings 47 wrapped around shaft 18 symmetrically about cylindrical axis 17 and proximate to distal portion 20. The plurality of flexible bristles 48 may be formed from silicone, rubber, plastic, and/or nylon, for example. In some embodiments, the plurality of flexible bristles 48 may be formed by molding shaft 18 concurrently with the plurality of flexible bristles 48.

FIG. 1E schematically illustrates a fifth embodiment of an apparatus 50 for extracting parasites, in accordance with some embodiments of the present invention. Apparatus 50 for extracting parasites may include shaft 18 with distal portion 20 configured for insertion through the anus into the lumen of a rectum of the subject. A proximal portion of shaft 18 may include handle 12. Guard portion 14, such as disk stopper 14, may be positioned on shaft 18 between handle 12 and distal portion 20 to prevent the distal portion from being inserted further than a predefined distance L into the rectal lumen. Distance L is typically on the order of 2.5-3 cm.

Shaft 18 in a region 53 at or near distal portion 20 may include a plurality of flexible protrusions such as a plurality of flexible bristles 52 attached symmetrically about cylindrical axis 17 to one or a plurality rings 54 wrapped around shaft 18 symmetrically about cylindrical axis 17 and proximate to distal portion 20. The plurality of flexible bristles 52 may be formed from silicone, rubber, plastic, and/or nylon, for example, which are soft and flexible when inserted and/or withdrawn from the rectum, yet rigid enough maintain contact with the rectal walls. In some embodiments, the plurality of flexible bristles 52 may be formed by molding shaft 18 concurrently with the plurality of flexible bristles 52. In some embodiments, the plurality of flexible bristles 52 and the one or a plurality of rings 54 may be formed in one integral unit.

In some embodiments of the present invention, the plurality of flexible protrusions shown in the multiple embodiments of FIGS. 1A-1E may be shaped to enhance the extraction and collection of the pinworms and/or pinworm eggs. For example, an inset 46 of FIG. 1E includes one bristle from the plurality of flexible bristles 52. Flexible bristle 52 may include a parasite collector tab 58 formed at a distal end and in the body of flexible bristle 52 as shown in inset 46. The shaping of the flexible bristles 52 in FIG. 1E may also be applied to flexible bristles 48 shown in FIG. 1D.

FIG. 1F schematically illustrates a sixth embodiment of an apparatus 60 for extracting parasites, in accordance with some embodiments of the present invention. Apparatus 60 for extracting parasites may include shaft 18 with distal portion 20 configured for insertion through the anus into the lumen of a rectum of the subject. A proximal portion of shaft 18 may include handle 12. Guard portion 14, such as disk stopper 14, may be positioned on shaft 18 between handle 12 and distal portion 20 to prevent the distal portion from being inserted further than a predefined distance L into the rectal lumen. Distance L is typically on the order of 2.5-3 cm.

Shaft 18 in a region 64 at or near distal portion 20 may include a plurality of grooves 62 symmetrically about cylindrical axis 17 and proximate to distal portion 20. The plurality of grooves 62 may be formed from silicone, rubber, plastic, and/or nylon, for example. In some embodiments, the plurality of grooves 62 may be formed by molding shaft 18 concurrently with the plurality of grooves 62. Any combination of groove sizes (e.g., length, width, for example) formed in the shaft may be used for each groove in the plurality of grooves so as to create a topology or texture along the surface of the shaft.

Pinworm eggs may stick to and/or be captured in the plurality of grooves 62 after insertion into the rectum via the anus. The plurality of grooves 62 may also be used to smash, smear and/or cut live pinworms during insertion into the rectum so as to kill the pinworms. Any suitable number of grooves 62 may be used to collect and/or extract pinworms and/or pinworm eggs. Apparatus 60 for extracting parasites may be stored and/or transported in a sterile protection cap 22 with lubricant gel and/or fluid 24 as previously described.

In various embodiments, the shaft may be molded to form the plurality of protrusions and/or the plurality of grooves used to extract the parasites. In various embodiments, the plurality of protrusions, the plurality of grooves, and the handle may be formed or fabricated externally and attached to the shaft by any suitable attachment process such as gluing, for example. In various embodiments, the shaft may be formed with any suitable texture where the formed texture may include grooves and/or protrusions configured to extract the parasites.

In some embodiments of the present invention, shaft 18 may include different colors and/or patterns so as not to frighten a child, for example.

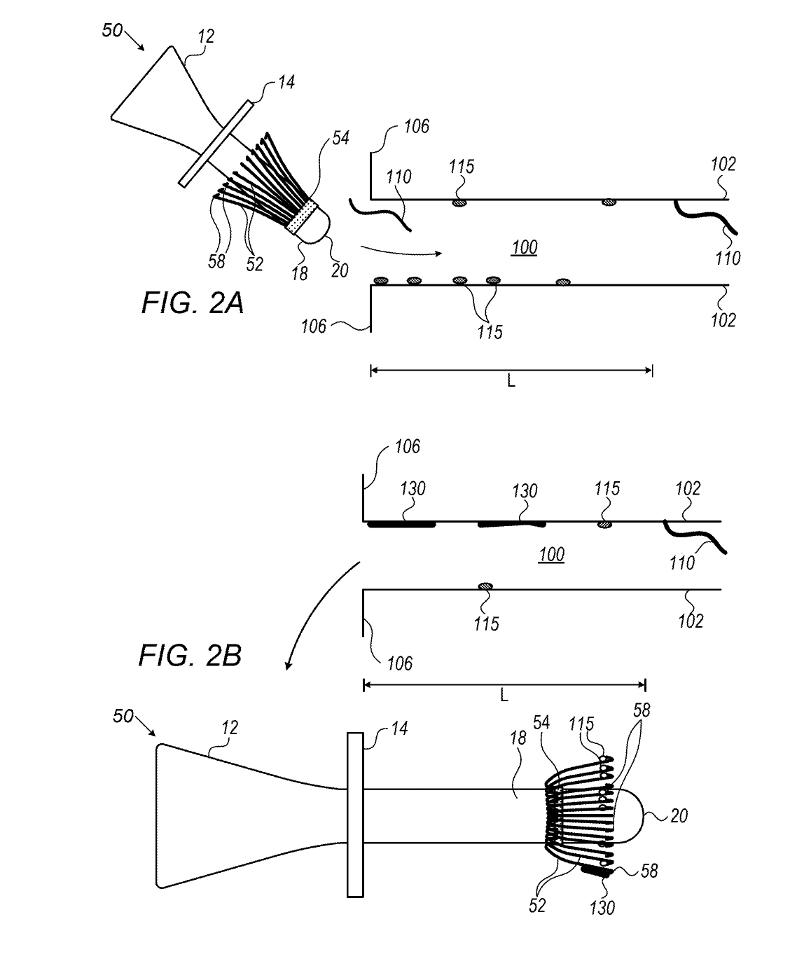

FIG. 2A schematically illustrates apparatus 50 for extracting parasites prior to insertion into a rectum 100, in accordance with some embodiments of the present invention. Live pinworms 110 and pinworm eggs 115 are shown in rectum 100. The entrance to the rectum is formed by external anal walls 106 which stop the disk stopper 14 so as to prevent shaft 18 from being inserted further than the predefined distance L into the rectum as described previously. Upon insertion of distal portion 20 of shaft 18 of apparatus 50 for extracting parasites into rectum 100, apparatus 50 for extracting parasites maintain the shape shown in FIG. 2A.

After apparatus 50 for extracting parasites is fully inserted up to disk stopper 14, the apparatus may then be withdrawn from the rectum. During withdrawal, rectal walls 102 may push upward on the plurality of bristles 52 causing the bristles to invert orientation about ring 54 where parasite collector tabs 58 may then be pushed into rectal wall 102 so as to collect pinworm eggs 115.

FIG. 2B schematically illustrates apparatus 50 for extracting parasites after withdrawal of apparatus 50 from rectum 100 with pinworm eggs 115 caught in parasite collector tab 58, in accordance with some embodiments of the present invention. After withdrawal, the plurality of bristles 52 are inverted in orientation about ring 54 with parasite collector tabs 58 facing upward (e.g., toward rectal walls when inside the rectum).

As parasite collector tab 58 contacts rectal walls 102 during extraction, parasite collector tab 58 may mush, smear and/or cut live pinworms 102 along rectal wall 102 and kill them as shown as dead pinworms 130. Additionally and/or alternatively, collected pinworm eggs and/or dead pinworms 130 or pieces thereof may be collected in parasite collector tab 58. Many of pinworm eggs 115 were removed by parasite collector tab 58 with some dead pinworms 130 remaining in rectum 100.

In some embodiments of the present invention, shaft 18 in the various embodiments of the apparatus for extracting parasites shown in FIGS. 1A-1F may be repeatedly inserted and withdrawn from rectum 100 so as to kill and reduce the number of live pinworms 110 which may lower the discomfort of the subject due to pruritus ani.

In some embodiments of the present invention, after the apparatus for extracting parasites extracts the parasites, the collected samples may be sent to a laboratory to assess if parasites are present in the subject. Typically, the apparatus for extracting parasites may be placed in a vial, such as a vial with a plastic or glass housing, for example. The vial may have a rectangular, square, elliptic or circular cross-section, for example. The vial may have any suitable form and/or shape.

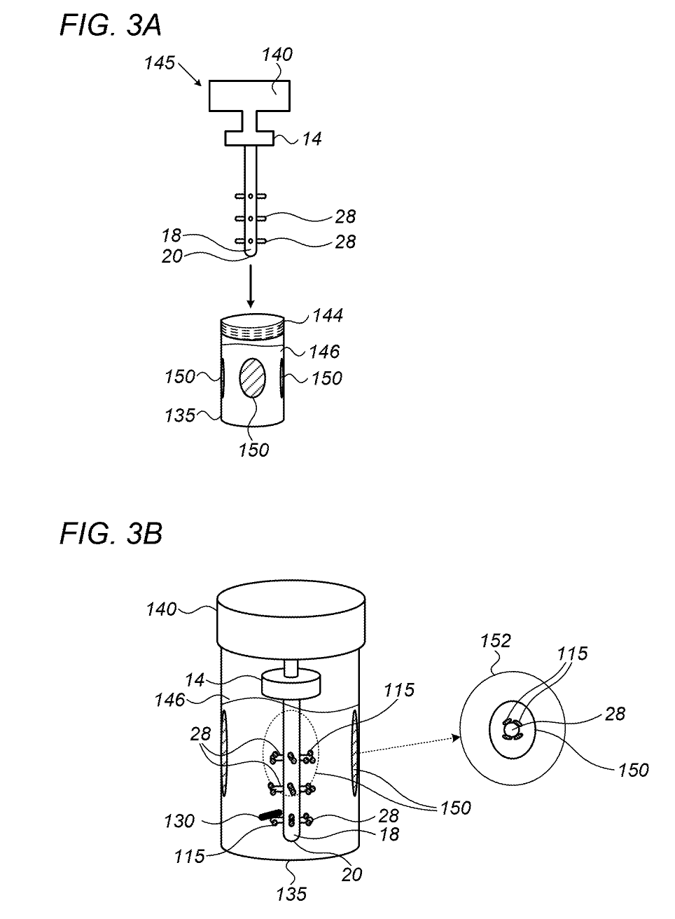

FIG. 3A schematically illustrates apparatus 145 for extracting parasites and a vial 135 for storing apparatus 145 after use, in accordance with some embodiments of the present invention. Apparatus 145 for extracting parasites includes a threaded cap 140 which is configured to be screwed into vial 135. Apparatus 145 for extracting parasites, and particularly flexible protrusions 28 are similar to those shown in FIG. 1B in this embodiment.

Vial 135 may be used storing apparatus 145 for extracting parasites after use. Threaded cap 140 may be configured to screw into threading 144 formed at the open end of vial 135. Vial 135 may include a fluid 146 for preserving the collected sample for pinworm detection.

In some embodiments of the present invention, vial 135 may include at least one lens 150, such as integrated lenses 150, for example, for observing pinworms and/or pinworm eggs collected on protrusions 28 after insertion of apparatus 145 for extracting parasites. The optical design of the integrated lenses 150 may utilize the shape of vial 135.

In some embodiments of the present invention, fluid 146 may include a color dye and/or stain for coloring the pinworms and/or the pinworm eggs, which are translucent. so as to enhance observation of the extracted parasites on apparatus 145 for extracting parasites using the at least one lens.

FIG. 3B schematically illustrates apparatus 145 for extracting parasites inserted into vial 135 after use, in accordance with some embodiments of the present invention. Integrated lenses 150 may formed on vial 135 and may be used to magnify so as to facilitate observing pinworms 110, dead pinworms 130 and/or pinworm eggs 115 on protrusions 28 as shown in an inset 152. FIGS. 3A and 3B illustrate apparatus 145 for extracting parasites similar to apparatus 25 for extracting parasites, which is merely used for visual clarity and not by way of limitation of the embodiments of the present invention. Any suitable apparatus for extraction parasites such as shown in FIGS. 1A-1F, for example, may be used in vial 135. In some embodiments of the present invention, fluid 146 may include a reagent such that when pinworms and/or pinworm eggs are present, the reagent may change color to indicate the presence of a pinworm infection. The reagent may include, for example, a Bradford colorimetric protein assay based on an absorbance shift of the dye Coomassie Brilliant Blue G-250. The red form of the dye may change to a bluer form binding to proteins in the pinworm and/or pinworm eggs.

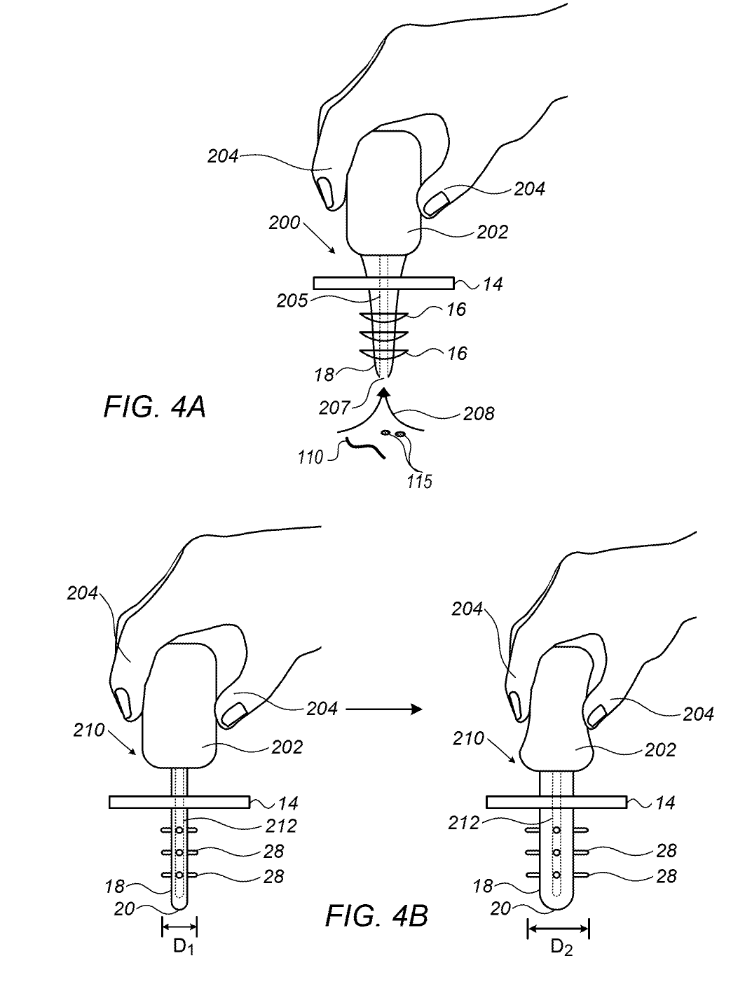

FIG. 4A schematically illustrates a seventh embodiment of an apparatus 200 for extracting parasites including a bulb 202, in accordance with some embodiments of the present invention. Bulb 202 may be attached to the proximal portion of shaft 18. Handle 12 may include bulb 202, or alternatively, bulb 202 may be in place of handle 12. Apparatus 200 for extracting parasites is similar to apparatus 10 for extracting parasites in FIG. 1A. However, shaft 18 may include a bore 205 through the longitudinal axis of shaft 18 (e.g., see longitudinal axis 17 in FIG. 1A) with an aperture 207 at the distal portion of shaft 18.

Fingers 204 of a user of apparatus 200 for extracting parasites may squeeze on bulb 202, may force out of all of the air inside bulb 202 and bore 205 through aperture 207, and may insert shaft 18 into the rectum of the subject. As shaft 18 is removed, the user may release his fingers 204 while aperture 207 may still be in the rectum, which may cause a vacuum 208 to form in the direction of aperture 207 which may draw parasites, such as pinworm eggs 115 through aperture 207 and into bore 205.

Thus, in addition to collecting pinworms and/or pinworm eggs on the plurality of flexible spherical caps 16, pinworms and/or pinworm eggs may be sucked into bore 205 and stored there. Bulb 202 may be formed from any suitable flexible material, such as rubber, for example. In some embodiments, only bore 205 may be used to collect pinworms and/or pinworm eggs without the shaft including a plurality of protrusions or a plurality of grooves.

FIG. 4B schematically illustrates an eighth embodiment of an apparatus 210 for extracting parasites including bulb 202, in accordance with some embodiments of the present invention. Bulb 202 may be attached to the proximal portion of shaft 18. Handle 12 may include bulb 202, or alternatively, bulb 202 may be in place of handle 12. Apparatus 210 for extracting parasites is similar to apparatus 25 for extracting parasites in FIG. 1B. However, shaft 18 may include a bore 212 through the longitudinal axis of shaft 18 (e.g., see longitudinal axis 17 in FIG. 1B).

Fingers 204 of a user of apparatus 210 for extracting parasites may squeeze on bulb 202 causing a change pressure within bore 212 in shaft 18. The changing pressure in closed bore 212 may cause a change in a diameter of the shaft. For example, the initial diameter of apparatus 210 for extracting parasites may be D₁. as shown in FIG. 4B. As bulb 202 is squeezed, the air pressure in bore 212 is increased. The shaft may be formed from flexible material and the increasing air pressure in the bore may increase the diameter of the shaft from D₁ to D₂, as shown in FIG. 4B. Shaft 18 may be formed from any suitable flexible material (e.g., rubber, plastic, nylon, silicone) which can expand or contract the diameter of shaft 18 under varying air pressure in bore 212 controlled by bulb 202.

The embodiments shown in FIGS. 4A and 4B are not limited to the shapes and types of flexible protrusions shown in FIGS. 4A and 4B. Any flexible protrusions and/or flexible grooves on shaft 18 may be used. Apparatus 210 for extracting parasites may be useful for controlling the contact force of the plurality of protrusions or the plurality of grooves with the rectal wall.

Different embodiments are disclosed herein. Features of certain embodiments may be combined with features of other embodiments; thus certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to he understood that the appended claims are intended to cover all such modifications and changes as fall within the true spirit of the invention.

1. An apparatus for extracting parasites from a rectum, the apparatus comprising:
a shaft with a distal portion configured to be inserted through an anus into the rectum, the distal portion of the shaft comprising a plurality of protrusions or a plurality of grooves designed to catch and hold parasites when removed from the rectum, the shaft further including a proximal portion designed to serve as a handle; and
a guard portion positioned on the shaft between the proximal portion and the distal portion of the shaft to prevent the distal portion from being inserted into the rectum further than a predefined distance.

2. The apparatus according to claim 1, wherein the plurality of protrusions or the plurality of grooves are designed to catch and hold parasites selected from the group consisting of pinworms and pinworm eggs.

3. The apparatus according to claim 1, wherein the plurality of protrusions comprise a plurality of spherical caps.

4. The apparatus according to claim 1, wherein the plurality of protrusions comprise a plurality of bristles.

5. The apparatus according to claim 4, wherein the plurality of bristles comprise a respective plurality of parasite collector tabs, and wherein extraction of the shaft from the rectum causes the plurality of parasite collector tabs to contact walls of the rectum and collect parasites.

6. The apparatus according to claim 1, wherein the plurality of protrusions are wound in a helical coil configuration around the shaft.

7. The apparatus according to claim 1, wherein the shaft, the plurality of protrusions, and the plurality of grooves are formed from materials consisting of silicone, rubber, plastic, and nylon.

8. The apparatus according to claim 1, wherein the shaft is molded to form the plurality of protrusions or the plurality of grooves.

9. The apparatus according to claim 1, further comprising a sterile cap, wherein the apparatus is configured to be stored in the sterile cap.

10. The apparatus according to claim 1, wherein plurality of protrusions or the plurality of grooves comprise a lubricant to ease insertion of the shaft into the rectum.

11. The apparatus according to claim 1, wherein the guard portion comprises a disk stopper.

12. The apparatus according to claim 1, wherein the guard portion is formed from a change in a diameter of the shaft.

13. The apparatus according to claim 1, further comprising a vial, and wherein the proximal portion of the shaft comprises a cap configured to be attached to an open end of the vial.

14. The apparatus according to claim 13, wherein the vial comprises a reagent which changes color to indicate a presence of extracted parasites in the plurality of protrusions or the plurality of grooves.

15. The apparatus according to claim 13, wherein the vial comprises at least one lens configured to magnify the extracted parasites in the plurality of protrusions or the plurality of grooves.

16. The apparatus according to claim 15, wherein the vial comprises a coloring dye for dying the extracted parasites in the plurality of protrusions or the plurality of grooves to enhance Observation of the extracted parasites using the at least one lens.

17. The apparatus according to claim 1, wherein the shaft including the plurality of protrusions or the plurality of grooves is configured to kill parasites in the rectum by repeatedly inserting and withdrawing the shaft from the rectum.

18. The apparatus according to claim 1, wherein the shaft comprises a texture that includes the plurality of protrusions or the plurality of grooves configured to extract the parasites.

19. The apparatus according to claim 1, further comprising a bulb attached to the proximal portion of the shaft, and wherein the bulb is configured to create a vacuum at an aperture of a bore formed in the shaft which draws parasites though the aperture into the bore.

20. The apparatus according to claim 1, further comprising a bulb attached to the proximal portion of the shaft, and wherein the bulb is configured to change pressure within a bore in the shaft, the changing pressure causing a change in a diameter of the shaft.
